(19) United States
(12) Patent Application Publication
(10) Pub. No.: US 2026/0203897 A1
(43) Pub. Date: Jul. 16, 2026

(54) METHOD FOR DETECTING A GENDER OF AN EMBRYONATED EGG, A RELATED COMPUTING DEVICE AND RELATED SYSTEM

(71) Applicant: PEGGY B.V., Gent (BE)

(72) Inventors: Jaroslav BELOTSERKOVSKY, Gent (BE); Jan VAN DEN BERGHE, Gent (BE); Geert TROOSKENS, Gent (BE); Christian DE WOLF, Gent (BE); Wim VAN CRIEKINGE, Gent (BE)

(21) Appl. No.: 19/138,157

(22) PCT Filed: Dec. 29, 2023
(86) PCT No.: PCT/EP2023/087995

§ 371 Date: Jun. 12, 2025

(30) Foreign Application Priority Data
Dec. 30, 2022 (BE) BE2022/6121

(51) Int. Cl. G06T 7/00; G01N 33/08

(52) U.S. Cl. CPC G06T 7/0012; G01N 33/085; G06T 2207/20081; G06T 2207/20084; G06T 2207/30044

(57) ABSTRACT

Embodiments of the invention relate to a Method for detecting viability of an embryonated egg, where said method comprises the steps of acquiring, by an image capturing device at least one image on an egg being illuminated by an illumination device (ID), wherein said method further comprises the steps of extracting at least one feature associated with anatomical structures of said egg from said at least one image, said anatomical structures of the egg comprising at least one of anatomical structures: air sac, yolk, the size or shape of the egg, the egg shell, shell membrane or other anatomical features of the egg, including such internal and external features and classifying a gender of an embryonated egg based on said at least one feature extracted associated with anatomical structures of said egg and an object recognition model generated comprising at least one feature characterizing each gender.

### TECHNICAL FIELD

Embodiments of the present invention relate to a method for detection of viability of unhatched eggs, and more particularly, to automated detection of gender of unhatched eggs using image data, to a related computing device and a related system.

### BACKGROUND

Several approaches have been used to determine the gender of fertilized eggs based on molecular and hormone assays that are laborious and invasive in nature. The techniques are of limited use in the industry as they are unsuitable for automated, high throughput applications.

Other approaches have used computer vision and spectroscopy to determine gender and/or fertility of unhatched eggs. However, such approaches have suffered from various drawbacks, including for example, poor performance on brown eggs, being limited in the data considered (e.g., limited to spatial data or limited to spectral data), being tested only on artificially fertilized eggs, etc. These methods are also applicable mostly at the late stage of embryo development, thus limiting their usefulness in the industry.

In Z. H. Zhu, Z. F. Ye, Y. Tang, “Nondestructive identification for gender of chicken eggs based on GA-BPNN with double hidden layers”, Journal of Applied Poultry Research, Volume 30, Issue 4, 2021, 100203, ISSN 1056-6171, https://doi.org/10.1016/j.japr.2021.100203 a machine vision image acquisition system is disclosed to identify the gender of chicken eggs at an early stage of incubation. In the process of image acquisition, the eggs to be detected were taken out from the incubator and placed horizontally for two minutes in order to let the blood vessels move into the upper side of the egg, and next images are acquired by a camera placed above the egg. The required settling time described is a bottleneck for the throughput of this analysis method, and furthermore it is unclear how robust this method is and whether it would be fit for application on an industrial scale.

Therefore, there is a need for improved technology for detecting gender and/or viability/fertility of embryonated eggs.

### DISCLOSURE OF THE INVENTION

In order to overcome the downsides of the prior art, there is proposed, according to a first aspect of the invention, a method for detecting a gender of an embryonated egg. Said method may comprise the following steps. As a first step, the embryonated egg may be illuminated. Next, image data is acquired of the embryonated egg being illuminated, wherein the image data is acquired from two or more viewpoints or viewing directions relatively to the embryonated egg. At least one feature associated with anatomical structures of said egg may be extracted from said at least one image data. A gender of the embryonated egg may be classified based on said at least one feature extracted associated with anatomical structures of said egg.

According to an embodiment of the first aspect, the anatomical structures of the egg comprise at least one of: air sac, yolk, the size or shape of the egg, the egg shell, shell membrane or other internal and/or external features.

According to an embodiment, illuminating the egg comprises providing an illumination device placed inside an egg holder holding the embryonated egg. This allows illuminating the egg with a light source outside the view of the capturing device. The illumination device is placed under the egg holder, providing radiation in a vertical direction. Images are subsequently captured in a generally horizontal direction. This allows providing radiation from one direction and capturing images at a second, generally perpendicular direction.

According to an embodiment of the first aspect, the classifying step is furthermore based on a generated object recognition model comprising at least one feature characterizing each gender; and/or wherein based on the classification of the gender of the embryonated egg the egg is processed further in a predetermined manner.

According to an embodiment of the first aspect, the image data comprises images, processed images, panoramic images or videos. Embodiments comprise processing the captured data into images. Embodiments comprise processing the images, e.g. by filtering or sharpening or contrast enhanced method. In a further step, panoramic images or videos are formed from the image data.

According to an embodiment of the first aspect, the image data is acquired using at least one image capturing device, and the method comprises image processing according to at least one of a cylindrical panorama method, an object-centric panorama method or a multi-viewpoint capture method;

In embodiments, the cylindrical panorama method comprises:
placing the egg in a fixed position;
rotating the at least one image capturing device around the egg, preferably in a horizontal plane, while keeping at least a part of the egg in a focal point of the at least one image capturing device;
acquiring image data while the at least one image capturing device rotates around the egg, ensuring sufficient overlap between the images in the image data; and
combining the different images in the image data together to obtain a panoramic image of the egg.

In embodiments, the object-centric panorama method comprises:
placing the at least one image capturing device in a fixed position;
rotating the egg using a rotation device;
acquiring image data while the egg rotates using the at least one image capturing device, ensuring sufficient overlap between the images in the image data; and
combining the different images in the image data together to obtain a panoramic image of the egg.

In embodiments, the multi-viewpoint capture method comprises:
positioning multiple image capturing devices around the egg, such that each of the multiple image capturing devices is configured to capture the egg from a different viewpoint;
placing the egg in a fixed position;
acquiring image data using the multiple image capturing devices, ensuring sufficient overlap between the images from each image capturing device in the image data; and
combining the different images in the image data together to obtain a panoramic image of the egg.

According to an embodiment of the first aspect, the step of classifying a gender of said embryonated egg is based on said at least one extracted feature, wherein said classifying is based on:
matching said at least one extracted feature associated with at least one anatomical structure of said egg with said image recognition model, the image recognition model comprising the at least one feature, associated with the at least one anatomical structure of the egg, the at least one feature characterizing a gender to obtain a degree of conformance; and
classifying the gender of the embryonated egg, based on the degree of conformance between the at least one extracted feature and said object recognition model comprising the at least one feature.

According to an embodiment of the first aspect, the image data comprises images, and wherein the number of acquired images is adapted based on a predetermined confidence level or a predefined time limit of the classification, such that the image data acquisition is actively adjusted by acquiring images until either the predetermined confidence level is obtained or the predefined time limit is reached.

In embodiments of the first aspect, any of the features disclosed herein, and in particular of the device according to the second aspect or the system of the third aspect, can be combined with this first aspect.

According to a second aspect of the invention, there is disclosed a computing device for detecting a gender of an embryonated egg. The computing device may comprise at least one of:
an image reception means, configured to receive image data of the embryonated egg being illuminated, wherein the image data is acquired from two or more viewpoints or viewing directions relatively to the embryonated egg;
an extraction means (EM), configured to extract at least one feature associated with anatomical structures of said egg from said received image data that comprises multiple images acquired from two or more viewpoints or viewing directions; and
a classification means (CLM), configured to classify a gender of an embryonated egg based on said at least one feature extracted associated with anatomical structures of said egg.

According to an embodiment of the second aspect, the computing device comprises image processing means configured to combine the different images in the image data together to obtain at least one panoramic image of the egg. Those panoramic images are subsequently processed in the EM.

According to an embodiment of the second aspect, the computing device further comprises a preprocessing means (PPM), configured to preprocess the images comprised in the image data, said preprocessing means (PPM), being configured to perform at least one of:
centering at least one of the images on said egg; and
resizing at least one of the images; and
removing a background of at least one of the images; and
optimizing a contrast of at least one of the images.

According to an embodiment of the second aspect, the classification means is configured to classify the gender based on a generated object recognition model comprising at least one feature characterizing each gender, preferably wherein the computing device is configured to generate the object recognition model based on training an initial object recognition model with a dataset comprising a plurality of images of illuminated eggs, revealing anatomical structures of the inner egg and applying a neural network with associated features characterizing an image.

According to an embodiment of the second aspect, the classification means (CLM) is configured: to match said at least one extracted feature associated with at least one anatomical structure of said egg with said image recognition model, the image recognition model comprising the at least one feature, associated with the at least one anatomical structure of the egg, the at least one feature characterizing a gender to obtain a degree of conformance; and to classify the gender of the embryonated egg, based on the degree of conformance between the at least one extracted feature and said object recognition model comprising the at least one feature.

In embodiments of the second aspect, any of the features disclosed herein, and in particular of the method according to the first aspect or the system of the third aspect, can be combined with the second aspect.

According to a third aspect of the invention, there is disclosed a system for detecting a gender of an embryonated egg. The system may comprise:
at least one illuminating means (ID) configured to illuminate an unhatched embryonated egg;
at least one image capturing device (ICD) configured to acquire image data of the illuminated egg; and
a computing device (CD) for detecting said gender of said embryonated egg based on the acquired image data.

In a preferred embodiment, the computing device (CD) is according to the second aspect of the invention.

According to an embodiment of the third aspect, the system further comprises actuation means (AM) configured to actuate a further action on said embryonated egg based on the classification by the classification means.

According to an embodiment of the third aspect, the system furthermore comprises an incubator, forming one or more incubation locations where the embryonated egg can be incubated, and a transportation system to transport the embryonated egg from the incubation location to an imaging location where the at least one image capturing device is configured to acquire an image of the egg and/or to transport the embryonated egg from the imaging location to the incubation location.

According to an embodiment of the third aspect, the image data comprises images, and wherein the number of acquired images is adapted based on a predetermined confidence level or a predefined time limit of the classification, such that the image data acquisition is actively adjusted by acquiring images until either the predetermined confidence level is obtained or the predefined time limit is reached.

In embodiments of the third aspect, any of the features disclosed herein, and in particular of the method according to the first aspect or the device of the second aspect, can be combined with the third aspect.

A further aspect of the invention is to provide a method, device or system, wherein image data comprises multiple images of an embryonated egg, and wherein the number of acquired images is adapted based on a predetermined confidence level or a predefined time limit of the classification, such that the image data acquisition is actively adjusted by acquiring images until either the predetermined confidence level is obtained or the predefined time limit is reached. This further aspect can be combined with any of the features disclosed herein.

An object of embodiments of the present invention is to provide a method for detecting gender of embryonated eggs, a related computing device and a related system of the above known type but wherein the aforementioned shortcoming or drawbacks of the known solutions are alleviated or overcome.

In particular it may be an additional objective of the present invention to provide with a method for detecting the viability/fertility of embryonated eggs, a related computing device and a related system of the above known type but wherein the aforementioned shortcoming or drawbacks of the known solutions are alleviated or overcome.

Indeed this objective is achieved by first acquiring at least one image of an illuminated egg and subsequently, extracting at least one feature that is associated with anatomical structures of the respective egg and finally classifying a sex of the embryo comprised in the subjected egg, based on said at least one feature extracted and a generated object recognition model (on a respective female embryonated egg and a male embryonated egg) comprising at least one feature characterizing each gender, i.e. at least one feature characterizing an unhatched egg comprising a female embryo and a set of at least one feature characterizing an unhatched egg comprising a male embryo.

The anatomical structures of the egg may comprise at least one (or any one or combination) of anatomical structures such as: air sac, yolk, the size or shape of the egg, the egg shell, shell membrane or other anatomical features of the egg, including such internal and external features.

The first step of acquiring an image on an illuminated egg may be performed by positioning the to be examined egg, preferably, in a dark environment, in a predefined position relative to at least one light source, such as a white light from any source such as an incandescent light bulb/lamps, LED or fluorescent lamps; single color (e.g. red, green, blue) light sources or application of color filters to block out specific wavelengths of light, or from LED or an Ultraviolet light or an infrared source. The illumination parameters may be adjustable or pre-selected, such as the color temperature of the light source, the luminoscity, and power. The plurality of images may be combined in a larger single PANO(ramic) image.

Furthermore, at least one image capturing device such as a Digital still photography camera with Charge Coupled Device CCD, complementary MOS sensor CMOS or similar sensors, digital videography cameras or similar is positioned in a further predefined position relative to the to be examined egg.

The capturing device may focus on the surface of the outer shell of the egg, on the center of the egg, on the inner egg or alternatively on the Air chamber/cell; yolk; albumen; embryo; allantois; amnion of the subject egg.

In a particularly preferred embodiment, the method additionally comprises the step of preprocessing said acquired image where said step of preprocessing may include at least one of:
centering said image on said egg; and
resizing said image acquired to a certain size suitable for processing in the generating of the object recognition model of the egg; and
removing a background of said image; and
optimizing a contrast of said image.

The pre-processing step may be based on an object detection algorithm and corresponding object recognition model that is configured to detect, by means of an object detection algorithm, the egg shape and/or embryo coordinates in the image, subsequently allowing to automatically centre, resize and/or skew and eliminate the background around the detected object e.g., including the egg or embryo.

In a still further preferred embodiment of the method, the step of removing said background of said at least one image is based on at first detecting an object in each said image, and subsequently the step comprising the isolating of said object detected from (the background) of said image by excising the detected object from the image or alternatively removing the background, i.e., the part of the image excluded from the detected object.

In a still further preferred embodiment of the method the step of extracting at least one feature associated with anatomical structures or features of the egg comprises the extracting of said at least one feature based on image recognition configured to detect at least one feature extracted associated with structures of the egg.

In a still further preferred embodiment of the method the step of generating said object recognition model is based on training an initial object recognition model with a dataset comprising a plurality of images of illuminated eggs, revealing anatomical structures of the egg applying a neural network such as for example a convolutional neural network wherein associated features characterizing an image. Based on the associated features of an image a gender can be determined.

In still another preferred embodiment of the method the step of classifying a gender of the embryo is based on said at least one feature extracted where said classifying is based on the first step of matching said at least one feature extracted, which feature is associated with anatomical structures of the egg, with said object recognition model comprising said at least one feature associated with anatomical structures of the subject egg including anatomical structures such as, air sac, yolk, the size and/or shape of the egg, the egg shell, shell membrane or other anatomical features of the egg, including such internal and external features characterizing each said gender to obtain a degree of conformance and subsequently the step of classifying said gender of embryonated egg, based on said degree of conformance between said at least one feature extracted associated with anatomical structures of the egg and said object recognition model comprising at least one feature associated with anatomical structures characterizing each gender. The outcomes of the step of classifying could be the following classes: female and male, alternatively be female, male, undetermined, alternatively be viable and non-viable.

In a further preferred embodiment of the method the step of classifying a gender of the embryo, the step of Acquiring, a first data set comprising said at least one image on an egg being illuminated by at least one illumination device (ID), a number of images of said first data set is adapted based on a predetermined performance level of a classifier in a predefined time window of acquiring said first data set. An important aspect of the invention is the real time implementation of the classifier allowing for progressive/adaptive sampling. This process enables the acquisition of additional (image) data in a maximal allowed time window if the initial data was not conclusive. The real time implementation allows for the classifier to require further images if the initial images resulted in an inconclusive classification, i.e., if the predetermined performance level was not reached. The performance level can be realized by a high enough confidence level in the acquired classification.

Mentioned adaptive real time sampling is a new method of collecting data where the data acquisition is actively adjusted based on the predetermined performance level of the classifier and predefined time window. The goal of adaptive sampling is to increase the confidence of the classifier in making a prediction by acquiring additional data.
1. At first, a classifier is trained on an initial set of complete/exhaustive image data.
2. The classifier is then used to make predictions on a set of test data (which variable subsampling) to test the performance as a function of data presented to the model
3. The classifier's performance on the test data is used to assess the performance increase obtained by adding additional data
4. The real-time implementation of the classifier continuously classifies the data until sufficient confidence is obtained or until data acquisition times out.

As an additional feature of the present invention, the deep learning algorithm can be initialized with prior validated retinal scan thereby leveraging gender specific vascularization.

Such Object recognition model is trained to take the acquired image of the egg as input and based hereon output one or more features or labels describing or characterizing the image. The set of possible output features or labels are referred to as target classes. The object recognition model is trained with a dataset comprising a plurality of images of illuminated eggs, revealing anatomical structures of the subject egg including anatomical structures such as: the air sac the yolk, the size and/or shape of the egg, the egg shell, shell membrane or other anatomical features of the egg, including such internal and external features.

As an additional feature, the Computing Device CD further may be configured, by means of an actuation means AM to actuate a further action on said embryonated egg based on said degree of conformance where such a further action may include causing the egg under test to be moved back to the incubator or hatchery, or follow another path to a further destination. The actuation means AM may be configured to be based on the determined degree of conformance and a corresponding class to have a different physical output process for each class of eggs wherein the output process could be to return to the incubator or hatchery or alternatively be the forwarding to a further destination.

### BRIEF DESCRIPTION OF THE DRAWINGS

The invention will be further elucidated by means of the following description and the appended figures.

FIG. 1 represents functional elements of a system for detecting gender of embryonated egg;

FIG. 2 represents the functional elements of the computing device according to embodiments of the present invention;

FIG. 3 represents a set of images captured on anatomical structures of an embryonated eggs;

FIGS. 4A-4C show the at least one image capturing device and the embryonated egg while performing respectively a cylindrical panorama method, an object-centric panorama method or a multi-viewpoint capture method when acquiring image data of the egg using the at least one image capturing device; and

FIG. 5 shows the combination of multiple images of the image data into a panoramic image.

### MODES FOR CARRYING OUT THE INVENTION

The description and drawings merely illustrate the principles of the invention. It will thus be appreciated that those skilled in the art will be able to devise various arrangements that, although not explicitly described or shown herein, embody the principles of the invention and are included within its spirit and scope. Furthermore, all examples recited herein are principally intended expressly to be only for pedagogical purposes to aid the reader in understanding the principles of the invention and the concepts contributed by the inventor(s) to furthering the art, and are to be construed as being without limitation to such specifically recited examples and conditions. Moreover, all statements herein reciting principles, aspects, and embodiments of the invention, as well as specific examples thereof, are intended to encompass equivalents thereof.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not necessarily correspond to actual reductions to practice of the invention.

Furthermore, the terms first, second, third and the like in the description and in the claims, are used for distinguishing between similar elements and not necessarily for describing a sequential or chronological order (or order of relative importance). The terms are interchangeable under appropriate circumstances and the embodiments of the invention can operate in other sequences than described or illustrated herein.

Moreover, the terms top, bottom, over, under and the like in the description and the claims are used for descriptive purposes and not necessarily for describing relative positions. The terms so used are interchangeable under appropriate circumstances and the embodiments of the invention described herein can operate in other orientations than described or illustrated herein.

The term “comprising”, used in the claims, should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

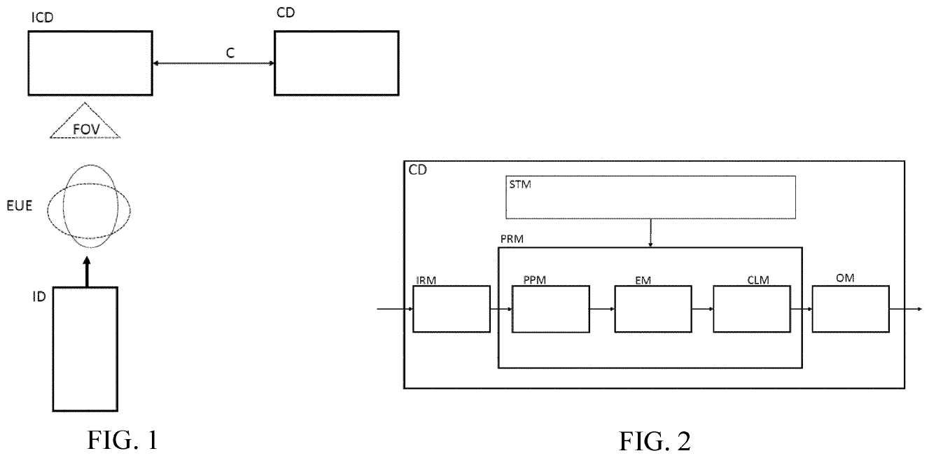

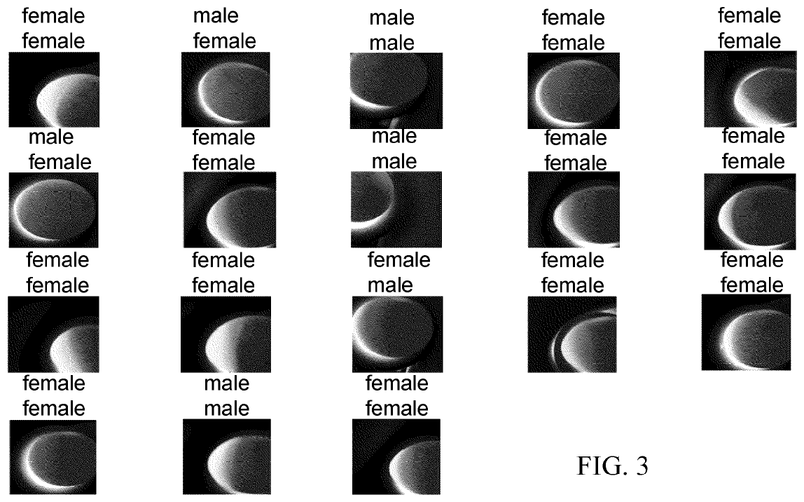

In the following paragraphs, referring to the drawing in FIG. 1, represents functional elements of a system for detecting viability and/or gender of an embryonated egg with the relevant elements of the system according to embodiments of the invention.

In a further paragraph, all connections between mentioned functional elements are defined.

Subsequently all relevant functional means of the computing device CD presented in FIG. 2 are described followed by a description of all interconnections.

In the succeeding paragraph the actual execution of the system is described.

The system for detecting the gender and/or the viability or the fertility of an embryonated egg first comprises at least one image capturing device ICD that is configured to acquire at least one image on the anatomical structures of an embryonated egg that is illuminated by an at least one illuminating device ID which illuminating device ID in turn is configured to illuminate an embryonated egg.

Furthermore, such an image capturing device ICD may be a Digital still photography camera with CCD/CMOS or similar sensors of a digital videography camera or similar which image capturing device ICD is positioned in a further predefined position relative to the to be examined egg so that the image acquired focuses on the appropriate part of the egg to be examined, i.e. focuses on the anatomical structures of the subject egg including anatomical structures such as: air sac, yolk, the size and/or shape of the embryonated egg, the egg shell, shell membrane or other anatomical features of the egg, including such internal and external features.

Alternatively, such image capturing device ICD may be at least one video camera capturing a plurality of images on the mentioned anatomical structures of the to be examined egg, i.e. the embryonated egg for which viability and/or the gender is to be determined. It is further to be noted that alternatively there could also be more than one camera and/or a plurality of light sources.

The at least one image capturing device ICD may be positioned relative to the egg in a fixed manner but alternatively also in a movable manner so that the anatomical structures of the egg to be captured by the image capturing device can be determined in a very flexible manner enabling a selection of structures to be captured in the at least one image. The at least one image capturing device ICD may move along a path around the, to be examined, egg in order to capture at least one still image or video (plurality of images) from the egg from all views or perspectives of the egg, i.e., 360 degrees view of the egg under examination, wherein the 360 degrees may be defined relative to any axis of a plurality of axes of the subject egg. The plurality of images may be combined in a larger single panoramic image.

The speed of movement of an at least one image capturing device ICD relative to the egg under test and or relative to the illumination device ID is of importance for enabling optimum quality of capturing images on anatomical structures of the egg under examination EUE preferably. It is to be understood that any one or all of the egg to be examined EUE, the at least one illuminating device UD, or the at least one image capture device ICD may be movable with respect to one another. For example, the EUE may be made to rotate along a predefined axis.

The optimum speed could lie in a range of one revolution (defined as a full revolution of 360 degrees) every 10 seconds, or 15 seconds, or 20 seconds.

In this way, not only the zooming in or zooming out on the anatomical structures of the to be examined egg can be performed but additionally the view on the to be examined egg can be determined: the image capturing device ICD can be positioned in such manner that the egg is captured in the long side perspective, in short side perspective in the top perspective, the bottom perspective or any mixed perspective of the to be examined egg.

The long side perspective is the view perpendicular to the long axis of the egg. The short side perspective is the view along (parallel to) the long axis of the egg.

The at least one image capturing device ICD could take a single image on the to be examined egg but preferably captures a plurality of images for enabling improved feature detection. The image data can comprise any combination of data that provides image data of the egg from multiple directions. In embodiments two images from opposite directions are provided as image data. In other embodiments, the image data can comprise video data of the egg, acquired from multiple directions.

The plurality of images may be combined in a larger single panoramic image. The combination of the plurality of images in a panoramic image renders the analysis of the combined image in the panoramic image more efficient due to the fact that a single panoramic image needs to be analysed instead of the plurality of distinct captures images.

Panoramic images are images with horizontally elongated fields of view. These images are also known as wide format image. An image showing a field of view approximating, or greater than, that of the human eye—about 160° by 75°—may be termed panoramic. This generally means it has an aspect ratio of 2:1 or larger, the image being at least twice as wide as it is high. The resulting images take the form of a wide strip. Some panoramic images have aspect ratios of 4:1 and sometimes 10:1, covering fields of view of up to 360 degrees. Both the aspect ratio and coverage of field are important factors in defining a true panoramic image.

In general, panoramic images typically have a wide field of view, often exceeding 100 degrees horizontally. Panoramic images are often created by stitching together multiple individual images. The number of images used and the degree of overlap between them can vary. Different projection methods may be used to map the three-dimensional scene onto a two-dimensional image. Common projections include cylindrical, spherical, and perspective projections.

In photography, the field of view (FOV) is that part of the world that is visible through the camera at a particular position and orientation in space; objects outside the field of view when the picture is taken are not recorded in the photograph. The field of view is most often expressed as the angular size of the view cone, as an angle of view. For a normal electronic camera, the diagonal (or horizontal or vertical) field of view can be calculated as:

$$FOV = 2 \times arctan ( \frac{sensor size}{2 f} )$$

where f is the focal length, here the sensor size and f are in the same unit of length, FOV is in radians.

If an image is made by combining several images, the field of view of that image can be determined by considering the field of view of each of the constituent images and their overlap. For example, if two images each have a field of view of 100 degrees and 10% overlap, the resulting combined image will have a field of view of 190 degrees.

In the present context, a panoramic image of an egg is a single, continuous image which shows details of the egg from a range of viewpoints in a viewing plane covering at least 90 degrees around the egg. In other words, the panoramic image can be formed from two or more images, which are taken (or look like they are taken from) two or more viewpoints in a viewing plane (for example, the horizontal plane) around the egg covering at least 90 degrees so that one can make these two images into a single, continuous image. Preferably, the range of viewpoints in the viewing plane cover at least 100 degrees around the egg, more preferably 120 degrees, even more preferably 180 degrees, even more preferably 270 degrees, most preferably 360 degrees.

Such a panoramic image can be obtained using multiple methods.

As a first method, one can rotate the camera in the viewing plane around the egg, while keeping the egg stationary. This method can be referred to as “cylindrical panorama.”

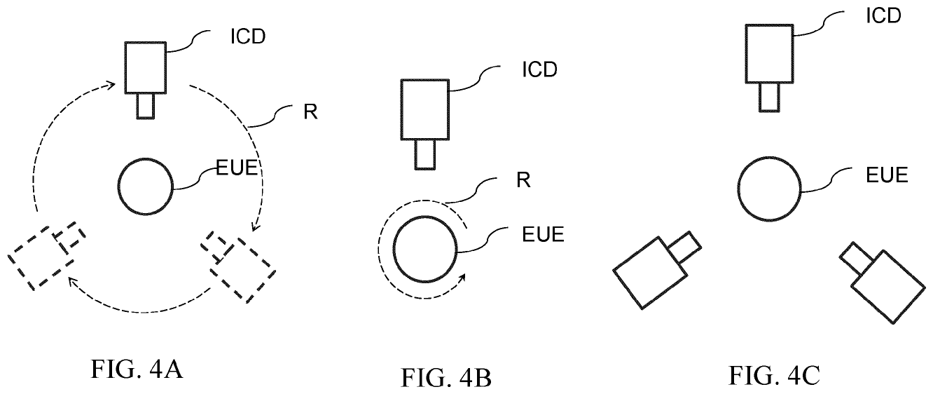

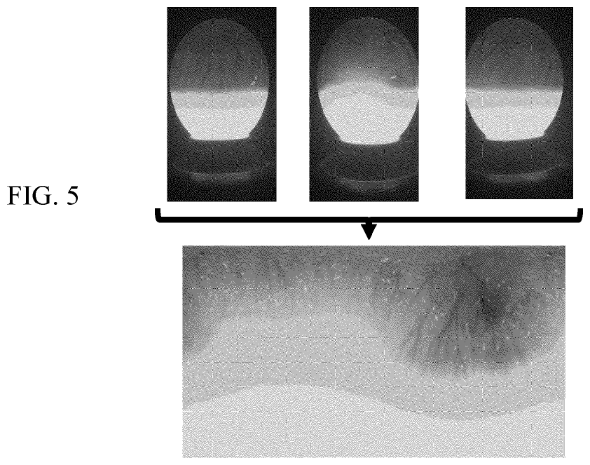

This method is shown in FIG. 4A. The image capturing device (ICD) is shown to rotate in a viewing plane around the egg under examination (EUE). The rotation is shown with dashed arrows R. The image capturing device is exemplary shown with dashed lines and full lines when the image capturing device acquires an image of the egg under examination.

For the setup, place the egg in a fixed position. Preferably, the egg is placed within an egg holder, such as a cup, such that the egg cannot fall or move during the image acquisition. In FIG. 5, a cylindrical cup holder is shown. In embodiments, the illumination device is positioned under the cup holder. The cup holder has a donut shape, open in the middle, allowing radiation to enter the egg via the bottom opening of the egg holder.

The camera is set up so that it can rotate around the egg, preferably keeping the surface of the egg or the inside of the egg in its focal point. Specialized panoramic tripod heads are for example available that facilitate precise rotation of the camera, further enhancing the stitching process of the different images. Next, rotate the camera horizontally around the egg, while taking pictures at (preferably regular) intervals to capture (part of) the entire circumference of the object.

Preferably there is sufficient overlap between consecutive images. This overlap can be important for accurate stitching during post-processing. Panoramic stitching software can be used to align and stitch/combine the individual images together. Any distortion or misalignments in the stitching process can be corrected in this step as well. The result can be a cylindrical panoramic image of the egg that provides a (partly or complete) horizontal view around the egg.

This method simplifies the stitching/combining process because the camera is rotating around a single point, reducing parallax issues. It is preferable that lighting conditions are consistent throughout the capture to avoid variations in exposure between images, otherwise one would need to correct for this in in pre- or post-processing. While the method was disclosed using a single viewing plane, the camera movement need not be confined to a two-dimensional plane.

As a second method, one can rotate the egg while keeping the camera in a fixed position. This method can be referred to as “object-centric panorama.”

This method is shown in FIG. 4B. The image capturing device ICD is shown at a fixed location, and acquires images of the egg under examination EUE that rotates around its rotation axis. The rotation is shown with a dashed arrow R.

For the setup, stably place the camera (e.g., mounted on a tripod) and place the egg on a rotation device. Next, rotate the egg using the rotation device, while keeping the camera stationary. Again, take pictures at (preferably regular) intervals or continuously (e.g., a movie) as the object rotates. The camera can capture at least (part of) the circumference of the egg, so as to obtain a panoramic image of the egg. Preferably the egg does at least one full rotation around its rotation axis. Again, sufficient overlap between consecutive images can be important for accurate stitching during post-processing. Similar post-processing methods can be used as in the cylindrical panorama method. The result can be a panoramic image of the egg that shows a (partly or complete) horizontal view around the egg.

This method helps minimize parallax errors, as the camera remains stationary. It is preferable that the egg is consistently and evenly rotated to avoid distortions in the final stitched image. Specialized rotation devices with precise control over rotation can be helpful for achieving optimal results. Control of the speed of rotation of the egg is important, a rotation that is too fast might result in the contents of the egg being moved around which could hamper proper imaging of the internal features of the egg, and could be detrimental to the livelihood of the egg. Preferably, the rotation device comprises an egg holder for holding the egg in place. The egg holder can comprise a cup or gripping arms. Additionally, lighting conditions are preferably uniform to prevent variations in exposure across the images.

As a third method, one can use multiple cameras set up around the egg at different angles. This method can be referred to as “multi-viewpoint capture.”

This method is shown in FIG. 4C. An exemplary number of three image capturing devices ICD are shown, which each acquire at least one image from an egg under examination EUE, from different viewpoints.

For the setup, position multiple cameras around the egg. Preferably, the egg is placed within an egg holder, such as a cup, such that the egg cannot fall or move during the image acquisition. Each camera captures the egg from its specific viewpoint. The cameras can be synchronized, or can obtain pictures asynchronously. The shorter the time period between each image from each camera, the higher the throughput (eggs analyzed per minute) of this method. Preferably, there is overlap between the fields of view of adjacent cameras to facilitate accurate stitching during post-processing. This post-processing can be similar to the post-processing of the cylindrical panorama method and the object-centric panorama method described above. The result can be a panoramic image that combines viewpoints from multiple cameras, providing a comprehensive view of the egg.

Using multiple cameras allows one to capture the object from various angles simultaneously, reducing the time required for image capture compared to a single camera. It may be important to calibrate the cameras to minimize distortion and achieve proper alignment during post-processing, so as to obtain a seamless and accurate final panoramic image. Preferably, consistent lighting conditions are used across all viewpoints.

In these three methods, the egg preferably is positioned with its major axis aligned with a vertical axis. The viewing plane is then preferably a horizontal plane. The egg can also be positioned with its major axis aligned with a horizontal axis, or at a different angle. Preferably, the major axis is substantially perpendicular to the viewing plane. The egg holder can be used to position and keep in place the egg.

At least one or multiple light sources can be used. For example, a light source can be placed within the egg holder, illuminating the egg from within the egg holder. Such a light source placed within the egg holder can be used with the three methods disclosed above, or any other suitable imaging method. A light source can also be (movably or stationary) placed opposite the (movable or stationary) camera. For example, if multiple cameras are used as in the multi-viewpoint capture method, each camera can have its own respective light source that it operates with. When one of the multiple cameras takes a picture, the attributed light source may be turned on and the other light sources may be turned off. A single light source can also be used, which is either stationary or which rotates along with the active cameras. The cameras might be positioned and/or configured in such a way that the image is not overexposed by the light source or that the camera does not take in light directly from the light source.

In these methods, the egg can be held and/or supported in an egg holder, such as a cup. The egg can be carefully placed within the egg holder, using e.g. robotic grippers or any other actuation means. An egg can also be placed in the egg holder by an operator by hand.

The use of panoramic images has as a benefit that a single image comprises all relevant viewpoints of the internal structure of the egg. That means that the analysis software only needs to process this singular image, having already been stitched together, so as not to look at the overlapping area more than once.

Furthermore, one does not have to wait until the internal structure of the egg settles into a stable position (determined by how the egg is positioned within the device, for example using the egg holder). This settling time can be a bottleneck for throughput. By taking an image from multiple viewpoints, and preferably stitching/combining them together into a panoramic image, the egg can be viewed from all sides and the optimal view of the internal structure of the egg can be determined and used in the analysis/processing, without waiting for the internal structure of the egg to settle into a stable position.

Furthermore, instead of stitching/combining the views from multiple viewpoints together into a panoramic image, it is also possible to (preferably automatically) select from these views obtained from the three methods above the best view for imaging (for example, a view where an internal structure of the egg is particularly visible, well-positioned or well-orientated) and to only perform the analysis/processing on this view. In this way, the image that needs to be analysed or processed is limited in size and more easily processed.

Instead of a picture, the one or more cameras can also perform video capture. The resulting video can be further analysed or processed. For example, stills from the video can be turned into a panoramic image, or one or multiple still from the video can be directly used, or (e.g., a part of) the video can be used directly.

In general, the image data (pictures, processed pictures (such as a panoramic image) or videos) can show the egg from two or more viewpoints or viewing directions relatively to the egg. In this way, more information of the egg is available than solely from a single viewpoint.

The resulting image data can then be analysed or processed to determine the gender of the egg.

FIG. 5 shows an exemplary combination of three images, each taken from a different viewpoint of the egg, which are combined into a single panoramic image showing the egg from two or more viewpoints or viewing directions relatively to the egg.

In the above examples, the two or more viewpoints were situated within the same viewing plane, but this is not generally required. The two or more viewpoints do not necessarily have to be located within a single viewing plane. For example, one viewpoint can look at the top of the egg, while other viewpoints can look at the side of the egg within a horizontal viewing plane.

Further, the meant at least one illuminating device ID being a light source, may comprise an Ultraviolet light source or alternately white light from any source such as an incandescent light bulb/lamps, LED or fluorescent lamps; single color (e.g., red, green, blue) light sources form LED; ultraviolet lamps.

The wavelength of the light of the meant light source can be of any one or combination of wavelengths in the visible, infrared, ultraviolet spectra. But preferably may be in a range of visible light.

The egg under examination (EUE) furthermore may be positioned still with respect to the Image capturing device (ICD) and illuminating device (ID) in a fixed position or may alternatively be positioned moveable with respect to the Image capturing device ICD and illuminating device ID for instance by means of a conveyor belt or rotating platform, alternatively by means of robotic actuators, motors or similar object manipulation devices.

The computing device may be a computing device dedicated for executing a software application for detecting the viability and/or the gender of an embryonated egg which software application may be executed at an incorporated processor, which computing device may be implemented at a local user device like a personal computer, mobile user computing device like a smartphone or alternatively be implemented at a (distributed) server system.

The Image capturing device ICD and the computing device are coupled over any wired or wireless connection such as an ethernet connection, a Bluetooth or Wi-Fi connection or other suitable connection.

The computing device CD further may incorporate or be coupled to a display and keyboard for interfacing with the computing device CD.

The meant computing device CD for detecting the viability or gender of an embryonated egg, as presented in FIG. 2 may comprise:
An image reception means IRM that is configured to receive at least one image acquired on the anatomical structure of an illuminated egg.

The computing device CD further may comprise a processing means PRM that in turn comprises:
An extraction means EM that is configured to extract at least one feature associated with the anatomical structures of said egg; and
A classification means CLM that is configured to classify a gender of said embryo based on said at least one feature extracted associated with said anatomical structures of said egg and an object recognition model generated comprising at least one feature characterizing each gender.

The processing means PRM of the computing device CD may further comprise a preprocessing means PPM that is configured to preprocess said acquired image, optionally based on an object recognition algorithm, where said preprocessing means PPM is configured to perform at least one of the following tasks:
Centering said at least one image on said egg; and
Resizing said at least one image; and
Removing a background of said at least one image; and
Optimizing a Contrast of said at least one image.

The preprocessing of the at least one acquired image impacts characteristics of the captured image to better distinguish features of the feature associated with anatomical structures of said egg and improve the extraction of these feature associated with anatomical structures of said egg.

The computing device additionally comprises a storage means STM that is configured to store data such as program data comprising the instructions to be executed by the processing means for performing the functionality of the processing means and furthermore the image data received by the image reception means IRM and all processed data resulting directly or indirectly from the processing of the image data, i.e., the feature extraction and the classification of the gender.

Furthermore, the computing device CD may comprise an output means OM configured to output results of the classification indicating the gender of the egg under examination and/or the viability or fertility of said embryo based on said at least one feature extracted associated with said anatomical structures of said egg and an object recognition model generated comprising at least one feature characterizing each gender.

Such computing device comprising the processing means PRM that may further comprise or be coupled to a storage device that can have program instructions stored thereon for causing a (micro) processor device to perform the instructions of the present invention. Accordingly, instructions for implementing embodiments of the present invention can be implemented in digital electronic circuitry, computer hardware, computer firmware, computer software, or any combination thereof. Instructions for implementing embodiments of the present invention can be implemented in a computer program product tangibly embodied in a machine-readable storage device for execution by a programmable processor so that the programmable processor executing program instructions can perform functions of the present disclosure. The instructions for implementing embodiments of the of the present invention can be implemented advantageously in one or more computer programs that are executable on a programmable system including at least one programmable processor coupled to receive data and instructions from, and to transmit data and instructions to, a data storage system, at least one input device, and at least one output device. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks (or any future equivalent). Any of the foregoing can be supplemented by, or incorporated in, ASICs (application-specific integrated circuits). Examples of such client device include, but are not limited to, are laptops, tablets and or smartphones (non-exhaustive list).

As a consequence, the mentioned means of the computing device may comprise any combination of hardware and/software for performing the defined functionality. The means may be implemented by means of a processor with coupled memory for storing the code of the application, data such as program data comprising the instructions to be executed by the processing means for performing the functionality of the processing means and furthermore the image data received by the image reception means IRM and all processed data resulting directly or indirectly from the processing of the image data, i.e., the feature extraction and the classification of the gender.

results of execution of the application and/or intermediary results of the application executed as presented in the embodiment application be by means of any combination of software and hardware.

This storage may be a memory located internally at the system or be located remotely at a single location or may be distributed memory, being a local database or a distributed storage system or any suitable alternative system or device.

The computing device CD may be a part of a computing system like a workstation as a desktop personal computer or a laptop personal computer or equally suitable device or be implemented at a server device being located locally or remotely from the user.

The image reception means IRM has an input-terminal that is an input terminal of the computing device CD. The image reception means IRM further is coupled with an output to an input of the extraction means EM that in turn is coupled to the classification means CLM that in turn is coupled to output means OM. Alternatively or additionally the preprocessing means PPM is coupled between the image reception means IRM and the extraction means EM. The Output means OM further has an output-terminal that is at the same time an output terminal of the computing device CD. The Storage means SM further is coupled to elements of the processing means PRM.

In order to explain the present invention, it is assumed that the first step of acquiring an image on an illuminated egg may be performed by positioning the to be examined egg, preferably in a dark environment, in a predefined position relative to a at least one light source, such as a white light from any source such as an incandescent light bulb/lamps, LED or fluorescent lamps; single color (e.g., red, green, blue) light sources from LED or an Ultraviolet light source.

Furthermore, an at least one image capturing device ICD such as a Digital still photography camera with CCD/CMOS or similar sensors; digital videography cameras or similar is positioned in a further predefined position relative to the to be examined egg.

The image capturing device ICD may be a light photography device a Digital still photography camera with CCD/CMOS or similar sensors; digital videography cameras or similar.

Moreover, as an important aspect of the image capturing device is the field of view FOV of the indicating the selection of the egg under examination to be included in the image to be captured. Another very important aspect of the image capturing device is the depth of field DoF being the distance between the nearest and furthest elements in a scene that appear to be “acceptably sharp” in an image. The distance between the camera and the first element that is considered to be acceptably sharp is called DoF near limit.

The capturing device may focus on the surface of the outer shell of the egg, on the center of the egg, or alternatively focus on the inner egg on the Air chamber/cell; yolk; albumen; embryo, the allantois or on the amnion of the subject egg.

At the same time a certain depth of field DoF is selected in a certain range between very narrow and wide, e.g., lying in range that covers the entire length or width of the egg under examination, depending on the relative position of the egg to the image capturing device ICD. The depth of field preferably covers ½ of the width of the egg when imaged on the long side perspective, or more preferably ¼ of the width of the egg when imaged on the long side perspective.

It is further assumed that the image capturing device ICD captures an image from the to be examined egg positioned, relative to the image capturing device ICD in a long side perspective (or alternatively, in short side perspective in the top perspective, the bottom perspective or any mixed perspective of the to be examined egg). The image capturing device ICD further selects the portion of relevance FOV of the anatomical structure of the egg under examination.

At the time of capturing the image from the to be examined egg positioned in described manner relative to the image capturing device, the at least one illumination device ID being a light source illuminates the egg under examination EUE. The light for illuminating the egg under examination EUE may have wavelength lying in a range of 380-750 nm or alternatively in the infrared or ultraviolet light wavelengths, and have a light-intensity lying in a range being sufficient for enabling the visualization of the structures of the egg under examination.

It is further to be noticed that sufficient contrast in the captured image is obtained between the structures of the egg, where the contrast depends on the illumination parameters and the light permeability of egg structures such as the variable thickness of e.g. egg shells

A further relevant aspect is the position of the egg under examination EUE relative to the ground plane due to the fact that the anatomical structures of the egg under examination are not perse solid but may have a certain liquidity or have a certain viscosity having as a consequence that the anatomical structures are not necessarily located at a fixed position within the egg but depend on the orientation of the egg relative to the mentioned ground plane.

Preferably the egg under examination is positioned such that the long axis of the egg is perpendicular to the ground plane (i.e. in a vertical position) while the camera ICD is located in manner that the image of the egg under examination comprises the long perspective of the egg under examination.

The illumination could be positioned in such manner that the illumination source is in the same plane (parallel) to the field of view of the image capturing device ICD (back-lit). Preferably, the illumination source is in a perpendicular plane to the field of view of the image capturing device ICD.

The perspective of the egg relative to the camera chosen determines the anatomical structures of the egg being present within the portion of relevance of FOV.

Alternatively, the image capturing device could be a video camera capturing a sequence of images on the egg under examination EUE in a still position or alternatively moving along a certain predefined path around the egg.

The image capturing device ICD could move along a predefined path around the egg, around the long axis of the egg, around the short axis of the egg or around a predefined further axis of the egg. Preferably the path of the image capturing device ICD travelled around the short axis of the egg (i.e. imaged on the long side perspective).

Subsequently, the image capturing device ICD forwards the at least one captured image by means of an image forwarding means of the image capturing device towards the image reception means IRM of the computing device CD over the connection C coupling the image forwarding means of the image capturing device ICD to the image reception means IRM of the computing device CD.

Subsequently, the extraction means EM of the processing means PRM extracts at least one feature that is associated with anatomical structures of said egg from said at least one image captured optionally applying an object recognition algorithm. The anatomical structure of the egg may comprise at least one of anatomical structures such as: air sac, yolk, embryo; allantois; amnion of the subject egg the size and/or shape, the egg shell, shell membrane or other anatomical features of the egg, including such internal and external features of the egg under examination. As mentioned before, the image may be an example of image data (pictures, processed pictures (such as a panoramic image) or videos) that can show the egg from two or more viewpoints.

Finally, the classification means CLM in turn classifies a gender of said embryo based on said at least one feature extracted which at least one feature is associated with the anatomical structures of the egg under examination an object recognition model generated which comprises at least one feature characterizing each gender.

Moreover, based on at least one feature extracted which at least one feature is associated with the anatomical structures of the egg under examination and an object recognition model generated which object recognition model comprises at least one feature characterizing the viability of the egg under examination this viability of the egg under examination can be determined or predicted.

The object recognition model has recognized features from images fed to the model for which a label gender, i.e., male or female is coupled or alternatively a label on the viability of the embryo.

Upon recognition of such feature maintained by the object recognition model the model may output the corresponding label comprising the gender or the viability of the meant egg.

The object recognition model can be continuously trained by feeding the false live births/hatchings or false gender in combination with the respective corresponding image into the object detection model in order to correct the classification of this respective observation.

In a further alternative or additional embodiment of the present invention the object recognition model may be further trained based on findings of the objection recognition algorithm feeding the object recognition model based on the types of images which are considered more relevant or useful than others.

In a particularly preferred embodiment, the preprocessing means PPM additionally is configured to perform the preprocessing said acquired image where said step of preprocessing may include at least one of:
centering said image on said egg; and
resizing said image acquired to a certain size suitable for processing in the generating of the object recognition model of the egg; and
removing a background of said image; and
optimizing a contrast of said image.

The preprocessing means PPM additionally is configured to perform the pre-processing based on an object detection algorithm and corresponding object recognition model that is configured to detect the egg shape and/or embryo coordinates in the image, subsequently allowing to automatically center, resize/skew and eliminate the background around the detected object e.g., being the egg/embryo.

Such object detection algorithm could be implemented by means of a python neural network or for instance a suitable equivalent neural network.

In a still further preferred embodiment, the preprocessing means additionally is configured to remove said background of said image based on at first detecting an object in said image, and subsequently the step comprising the isolating of said object detected from the background of said image by excising the detected object from the image or alternatively removing the background, i.e., the part of the image excluded from the detected object.

The step of removing said background of said image is based on first detecting an object in said image acquired (optionally) based on an object detection model and subsequently isolating of said object from of said image acquired. The meant object is recognized based on an available object present in an object database.

The processing means PRM extracts the at least one feature associated with anatomical structures of the egg where the extracting of said at least one feature based on image recognition configured to detect at least one feature extracted associated with anatomical structures of the egg.

In a still further preferred embodiment, the processing means PRM additionally is configured to generate said image/object recognition model is based on training an initial object recognition model with a dataset comprising a plurality of images of illuminated eggs, revealing anatomical structures of the egg applying a neural network such as for example a neural network or a convolutional neural network. The anatomical structures of the egg may comprise structures such as: air sac, yolk, albumen; embryo; allantois; amnion of the subject egg the size and/or shape, the egg shell, shell membrane or other anatomical features of the egg, including such internal and external features of the egg under examination. The image/object recognition model is subsequently stored and maintained at the storage means STM of the computing device CD.

As an example, a dataset consisting of a plurality of images in an example 201 images whereof 138 female and 63 males, from 29 unique eggs is applied. In a first step, these images for instance may be centered on the egg by an object detection model as executed by the preprocessing means and subsequently resized to 336×336 px by the preprocessing means. Subsequently, as an embodiment of the present invention, on this set of images an object recognition model was trained e.g., by means of the object recognition algorithm such as a pytorch model on top of a pretrained resnet36 model.

Optimal learning rate was determined and model weights optimized using multiple learning cycles.

Such an object recognition model can be continuously be trained by feeding image data with confirmation data on the features comprised in the captured image. The object recognition model can be continuously be trained by feeding particularly based on the false live births/hatchings.

In a still another preferred embodiment the preprocessing means additionally is configured to classify a gender of the embryo based on said at least one feature extracted where said classifying is based on first matching said at least one feature extracted associated with anatomical structures of the egg with said object recognition model which comprises said at least one feature associated with anatomical structures of the subject egg including anatomical structures such as: air sac, yolk (which is not really part of the embryo), the size and/or shape of the egg characterizing each said gender. By matching said at least one feature extracted associated with anatomical structures of the egg with said object recognition model which comprises said at least one feature associated with anatomical structures of the subject egg a certain degree of conformance is obtained and subsequently the classification of said gender of the embryonated egg, is based on said degree of conformance between said at least one feature extracted associated with anatomical structures of the egg and said object recognition model comprising at least one feature associated with anatomical structures characterizing each gender.

Such Object recognition model is trained to take the acquired image of the egg as input and output one or more features or labels describing the image. The set of possible output features or labels are referred to as target classes. The object recognition model is trained with a dataset comprising a plurality of images of illuminated eggs, revealing anatomical structures of the subject egg including anatomical structures such as: the air sac the yolk, albumen; embryo; allantois; amnion of the subject egg the size and/or shape, the egg shell, shell membrane or other anatomical features of the egg, including such internal and external features of the egg under examination.

Such Object recognition model could be a python-based object recognition model.

As an additional feature, the Computing Device CD further may be configured, by means of an actuation means AM to actuate a further action on said embryonated egg based on said degree of conformance where such a further action may be causing the egg under test to be moved back to the hatchery, or follow another path to a further destination. The actuation means AM may be configured to be based on the determined degree of conformance and a corresponding class to have a different physical output process for each class of eggs wherein the output process could be to return to the hatchery or forwards to a further destination.

The output of the classification means CLM may comprise two or three classes where a first class may be the female embryo, a second class be the male embryo and an optional third class be indefinite meaning that the gender could not be determined with sufficient certainty.

Subsequently, based on the different physical output classes as outcome of the process, for each class of eggs (note: there may be three if ‘unsure’ is a separate class): an egg under examination EUE may be sent back to hatchery versus destroyed or moved to another location for further processing.

All of the mentioned means of the system may be incorporated in a machine that, at a pre-set legally allowed time and for breeding where the sex of the chick is important, scans all the eggs in a hatchery in an automated way, wherein every egg goes on a conveyor belt (in the right temperature and within the hatching parameters), gets its photo(s) taken and performs a classification of each egg under examination EUE. The computer-generated classification can be applied immediately in a subsequent action, and such action may include that the egg does or does not go back into the same hatchery.

To transport the egg from an incubation location to the image location of the image capturing device of the system for detecting a gender of the egg, a transportation system can be used. For example, a conveyor system with specially designed egg carriers can transport eggs from the incubation location to the imaging location. A conveyor system can be configured to maintain a gentle and steady movement, preventing jarring or vibrations that might disrupt the embryos. As another example, articulated robotic arms equipped with specialized grippers can be configured to carefully pick up and transfer individual eggs from the incubation location to the imaging location. The robotic arms can be designed with sensors to ensure a secure grip without applying excessive pressure. As another example, automated egg trays which comprise trays or holders with automated mechanisms can be integrated into both the incubator and the system for detecting a gender of the egg. The trays are designed to securely cradle individual eggs during transportation, ensuring stability and minimizing the risk of damage. As another example, pneumatic systems can be used that comprise controlled air pressure systems which can be employed to lift and transport eggs from one location to another. These systems can be programmed to provide a gentle and controlled airflow, lifting the eggs by creating a suction force. without causing any harm to the egg. Other exemplary methods are also possible. All these examples of transportation systems can be combined in an appropriate manner.

Note that the system for detecting a gender of the egg can be formed within the incubator, or the incubator can be formed within the system for detecting a gender of the egg. The system for detecting a gender of the egg and the incubator can also be formed separately, as stand-alone or connected units.

Preferably, the eggs in the incubator are stored in the same orientation as the orientation of the egg when acquiring the image using the image capturing device of the system for detecting a gender of the egg. The transportation system may likewise keep the egg in the same orientation during transport from the incubation location to the image location. In this way, the orientation of the egg can be kept constant. This may be beneficial in two ways. First of all, the egg is disturbed less since the orientation is kept the same, which is good for the livelihood of the egg. Secondly, because the structures within the egg may move around when changing the orientation of the egg, the structures within the egg are kept at the same location. This may increase the quality of the resulting images since structures may be reproducibly in the same location. Preferably, the eggs in the incubation location, in the image location and during transport are kept with their long axis pointing in a substantially vertical direction.

As an important note, it is to be remarked that the method according to any embodiment of the present invention is preferably to be executed or implemented before the 7th day of incubation (following laying), and more preferably on the 6th day.

A final remark is that embodiments of the present invention are described above in terms of functional blocks. From the functional description of these blocks, given above, it will be apparent for a person skilled in the art of designing electronic devices how embodiments of these blocks can be manufactured with well-known electronic components. A detailed architecture of the contents of the functional blocks hence is not given.

While the principles of the invention have been described above in connection with specific apparatus, it is to be clearly understood that this description is merely made by way of example and not as a limitation on the scope of the invention, as defined in the appended claims.

In the following, clauses are provided that provide further embodiments. Any of the features disclosed in the clauses can be combined with features according to the aspects disclosed herein.

Clause 1. Method for detecting a gender of an embryonated egg, said method comprising the steps of:
Acquiring, by at least one image capturing device (ICD) a first data set comprising at least one image on an egg being illuminated by at least one illumination device (ID), wherein said method further comprises the steps of:
Extracting, by a computing device, at least one feature associated with anatomical structures of said egg from said at least one image, said anatomical structures of the egg comprising at least one of anatomical structure: air sac, yolk, the size or shape of the egg, the egg shell, shell membrane or other anatomical features of the egg, including such internal and external features; and
Classifying, by a computing device, a gender of an embryonated egg based on said at least one feature extracted associated with anatomical structures of said egg and an object recognition model generated comprising at least one feature characterizing each gender.
   Clause 2. Method according to clause 1, wherein said method additionally comprises the step of preprocessing said at least one image acquired, said step of preprocessing may include at least one of:
Centering said at least one image on said egg; and
Resizing said at least one image; and
Removing a background of said at least one image; and
Optimizing a Contrast of said at least one image.
   Clause 3. Method according to clause 2, wherein said step of removing said background of said image is based on first detecting an object in said image acquired optionally based on an object detection model and subsequently isolating of said object from of said image acquired.
   Clause 4. Method according to any of the previous clauses, wherein said step of extracting at least one feature associated with said structures of said egg comprises the extracting of said at least one feature based on image recognition configured to detect at least one feature extracted associated with said anatomical structures of said egg.
   Clause 5. Method according to any of the previous clauses, comprising the step of generating said object recognition model based on training an initial object recognition model with a dataset comprising a plurality of images of illuminated eggs, revealing anatomical structures of the egg applying a neural network with associated features characterizing an image.
   Clause 6. Method according to any of the previous clauses, wherein said step of Classifying a gender of said embryonated egg is based on said at least one feature extracted where said classifying is based on:
Matching said at least one feature extracted associated with anatomical structures of said egg with said image recognition model comprising said at least one feature associated with said anatomical structures of said egg characterizing each said gender to obtain a degree of conformance; and
Classifying said gender of embryonated egg, based on said degree of conformance between said at least one feature extracted associated with anatomical structures of the egg and said object recognition model comprising at least one feature associated with anatomical structures of said egg characterizing each gender.
   Clause 7. Method for detecting a gender of an embryonated egg according to any of the previous clauses, wherein said step of Acquiring, a first data set comprising said at least one image on an egg being illuminated by at least one illumination device (ID), a number of images of said first data set is adapted based on a predetermined performance level of a classifier in a predefined time window of acquiring said first data set.
   Clause 8. Computing Device (CD) for detecting a gender of an embryonated egg, said device comprising:
An image reception means (IRM), configured to receive a first data set comprising at least one image acquired on an illuminated egg; wherein said device (CD) further comprises:
An extraction means (EM), configured to extract at least one feature associated with anatomical structures of said egg from said at least one image, said anatomical structures of the egg comprising at least one of anatomical structure: air sac, yolk, the size or shape of the egg, the egg shell, shell membrane or other anatomical features of the egg, including such internal and external features; and
A classification means (CLM), configured to classify a gender of said embryo based on said at least one feature extracted associated with anatomical structures and an object recognition model generated comprising at least one feature characterizing each gender.
   Clause 9. Computing Device (CD) for detecting a gender of an embryonated egg, according to clause 8, wherein said device further comprises a preprocessing means (PPM), configured to preprocess said acquired image, said preprocessing means (PPM), being configured to perform at least one of:
Centering said at least one image on said egg; and
Resizing said at least one image; and
Removing a background of said at least one image; and
Optimizing a Contrast of said at least one image.
   Clause 10. Computing Device (CD) according to clause 8 or 9, wherein said extraction means (EM), is configured to extract said at least one feature based on image recognition configured to detect at least one feature extracted associated with said anatomical structures of said egg.
   Clause 11. Computing Device (CD) according to any of the clauses 8-10, wherein said preprocessing means (PPM) is configured to remove said background of said image is based on first detecting an object in said image acquired optionally based on an object detection model and additionally configured to isolate said object from of said image acquired.
   Clause 12. Computing Device (CD) according to any of the clauses 8-11, wherein said computing device is configured to generate said object recognition model based on training an initial object recognition model with a dataset comprising a plurality of images of illuminated eggs, revealing anatomical structures of the inner egg applying a neural network with associated features characterizing an image.
   Clause 13. Computing Device (CD) according to any of the clauses 8-12, wherein said classification means (CLM) for classifying a gender of said embryonated egg based on said at least one feature extracted is configured to:
Match said at least one feature extracted associated with anatomical structures of said egg with said object recognition model comprising said at least one feature associated with said anatomical structures of said egg characterizing each said gender to obtain a degree of conformance; and further configured to:
Classify said gender of embryonated egg, based on said degree of conformance between said at least one feature extracted associated with anatomical structures of the egg and said object recognition model comprising at least one feature associated with anatomical structures characterizing each gender.
   Clause 14. Computing Device (CD) for detecting a gender of an embryonated egg, according to any of the clauses 8-13, wherein said Computing Device (CD) further comprises an actuation means (AM) configured to actuate a further action on said embryonated egg based on said degree of conformance.
   Clause 15. Computing Device (CD) for detecting a gender of an embryonated egg, according to any of the clauses 8-14, wherein said computing device further comprises means to combine said at least one image acquired in a panoramic image.
   Clause 16. System for detecting a gender of an embryonated egg, said system comprising at least one image capturing device (ICD) configured to acquire an image on an illuminated egg, at least one illuminating means (ID) configured to illuminate an unhatched embryonated egg and a computing device (CD) for detecting said gender of said embryonated egg, the computing device (CD) according to any one of clause 8 to clause 15.

1. Method for detecting a gender of an embryonated egg, said method comprising the steps of:
illuminating the embryonated egg;
acquiring image data of the embryonated egg being illuminated, wherein the image data is acquired from two or more viewpoints or viewing directions relatively to the embryonated egg;
extracting at least one feature associated with anatomical structures of said egg from said image data; and
classifying a gender of the embryonated egg based on said at least one feature extracted associated with anatomical structures of said egg.

2. The method of claim 1, wherein the anatomical structures of the egg comprise at least one of: air sac, yolk, the size or shape of the egg, the egg shell, shell membrane or other internal and/or external features.

3. The method of claim 1 or 2, wherein the classifying step is furthermore based on a generated object recognition model comprising at least one feature characterizing each gender; and/or wherein based on the classification of the gender of the embryonated egg the egg is processed further in a predetermined manner.

4. The method of any one of the preceding claims, wherein the image data comprises images, processed images, panoramic images or videos.

5. The method of any one of the preceding claims, wherein the image data is acquired using at least one image capturing device, by at least one of a cylindrical panorama method, an object-centric panorama method or a multi-viewpoint capture method;
wherein the cylindrical panorama method comprises:
placing the egg in a fixed position;
rotating the at least one image capturing device around the egg, preferably in a horizontal plane, while keeping at least a part of the egg in a focal point of the at least one image capturing device;
acquiring image data while the at least one image capturing device rotates around the egg, ensuring sufficient overlap between the images in the image data; and
combining the different images in the image data together to obtain a panoramic image of the egg;
wherein the object-centric panorama method comprises:
placing the at least one image capturing device in a fixed position;
rotating the egg using a rotation device;
acquiring image data while the egg rotates using the at least one image capturing device, ensuring sufficient overlap between the images in the image data; and
combining the different images in the image data together to obtain a panoramic image of the egg; and
wherein the multi-viewpoint capture method comprises:
positioning multiple image capturing devices around the egg, such that each of the multiple image capturing devices is configured to capture the egg from a different viewpoint;
placing the egg in a fixed position;
acquiring image data using the multiple image capturing devices, ensuring sufficient overlap between the images from each image capturing device in the image data; and
combining the different images in the image data together to obtain a panoramic image of the egg.

6. The method of any one of the preceding claims, wherein step of classifying a gender of said embryonated egg is based on said at least one extracted feature, wherein said classifying is based on:
matching said at least one extracted feature associated with at least one anatomical structure of said egg with said image recognition model, the image recognition model comprising the at least one feature, associated with the at least one anatomical structure of the egg, the at least one feature characterizing a gender to obtain a degree of conformance; and
classifying the gender of the embryonated egg, based on the degree of conformance between the at least one extracted feature and said object recognition model comprising the at least one feature.

7. The method of any one of the preceding claims, wherein the image data comprises images, and wherein the number of acquired images is adapted based on a predetermined confidence level or a predefined time limit of the classification, such that the image data acquisition is actively adjusted by acquiring images until either the predetermined confidence level is obtained or the predefined time limit is reached.

8. A computing device for detecting a gender of an embryonated egg, said device comprising:
an image reception means, configured to receive image data of the embryonated egg being illuminated, wherein the image data is acquired from two or more viewpoints or viewing directions relatively to the embryonated egg;
an extraction means (EM), configured to extract at least one feature associated with anatomical structures of said egg from said at least one panoramic image; and
a classification means (CLM), configured to classify a gender of an embryonated egg based on said at least one feature extracted associated with anatomical structures of said egg.

9. The computing device of claim 8, wherein the computing device further comprises image processing means configured to combine image data from two or more viewpoints together to obtain at least one panoramic image of the egg,
wherein preferably the image reception means are configured to receive image data of the embryonated egg being illuminated, wherein the image data contains multiple images that are acquired from two or more viewpoints,
wherein preferably said computing device further comprises a preprocessing means (PPM), configured to preprocess the images comprised in the image data, said preprocessing means (PPM), being configured to perform at least one of:
centering at least one of the images on said egg; and
resizing at least one of the images; and
removing a background of at least one of the images; and
optimizing a contrast of at least one of the images.

10. The computing device of claim 8 or 9, wherein the classification means is configured to classify the gender based on a generated object recognition model comprising at least one feature characterizing each gender, preferably wherein the computing device is configured to generate the object recognition model based on training an initial object recognition model with a dataset comprising a plurality of images of illuminated eggs, revealing anatomical structures of the inner egg and applying a neural network with associated features characterizing an image.

11. The computing device of claim 10, wherein the classification means (CLM) is configured to:
match said at least one extracted feature associated with at least one anatomical structure of said egg with said image recognition model, the image recognition model comprising the at least one feature, associated with the at least one anatomical structure of the egg, the at least one feature characterizing a gender to obtain a degree of conformance; and
classify the gender of the embryonated egg, based on the degree of conformance between the at least one extracted feature and said object recognition model comprising the at least one feature.

12. A system for detecting a gender of an embryonated egg, the system comprising:
at least one illuminating means (ID) configured to illuminate an unhatched embryonated egg;
at least one image capturing device (ICD) configured to acquire image data of the illuminated egg;
and a computing device (CD) for detecting said gender of said embryonated egg based on the acquired image data, wherein the computing device (CD) is according to any one of claims 8 to 11.

13. The system according to claim 12, further comprising actuation means (AM) configured to actuate a further action on said embryonated egg based on the classification by the classification means.

14. The system according to claim 12 or 13, wherein the system comprises an incubator, forming one or more incubation locations where the embryonated egg can be incubated, and a transportation system to transport the embryonated egg from the incubation location to an imaging location where the at least one image capturing device is configured to acquire an image of the egg and/or to transport the embryonated egg from the imaging location to the incubation location.

15. The system according to any one of claims 12-14, wherein the image data comprises images, and wherein the number of acquired images is adapted based on a predetermined confidence level or a predefined time limit of the classification, such that the image data acquisition is actively adjusted by acquiring images until either the predetermined confidence level is obtained or the predefined time limit is reached.
